(19) United States
(12) Patent Application Publication
(10) Pub. No.: US 2006/0275510 A1
(43) Pub. Date: Dec. 7, 2006

(54) Formulations for menopausal syndromes

(75) Inventors: Yongzheng Hui, Shanghai (CN); Quanzhong Sun, Shanghai (CN); Guoshun Tang, Shanghai (CN); Yuan Wang, Shanghai (CN); Weiyong Wu, Shanghai (CN); Zhen Yu Chen, Ma On Shan (HK); Lai Kwok Leung, Shatin (HK); Leung Franky Chan, Ma On Shan (HK); Jun Wang, Fotan (HK)

Correspondence Address: FISH & RICHARDSON P.C., PO BOX 1022, MINNEAPOLIS, MN, 55440-1022, US

(73) Assignee: Hong Kong Jockey Club Institute of Chinese Medicine Limited, Shatin (HK)

(21) Appl. No.: 11/202,550

(22) Filed: Aug. 11, 2005

(30) Foreign Application Priority Data
Jun. 3, 2005 (CN) 200510074259.1

(51) Int. Cl. A61K 36/484; A61K 36/48; A61K 36/43; A61K 36/53

(52) U.S. Cl. 424/725; 424/757; 424/745

(57) ABSTRACT

The present invention relates to the use of Chinese herbs Shu Di Huang, Yin Yang Huo, Shan Zhu Yu, Shan Yao, Gou Qi Zi, and Dan Dou Chi in preparation formulations for the treatment of menopausal syndromes and related symptoms.

### CROSS-REFERENCE TO RELATED APPLICATION

This application claims priority to Chinese Application No. 200510074259.1, filed Jun. 3, 2005, which is hereby incorporated by reference in its entirety.

### FIELD OF INVENTION

The present invention relates to the field of dietary supplements and therapeutic compounds. In particular, the present invention relates to formulations comprising herbal extracts for the amelioration of menopausal syndromes.

### BACKGROUND OF INVENTION

References which are cited in the present disclosure are not necessarily prior art and therefore their citation does not constitute an admission that such references are prior art in any jurisdiction.

Menopausal syndrome includes symptoms associated with the physiological changes that take place in a woman's body as her period of fertility ends. It affects most commonly women in the age-group between 45 to 55. Menopause is a normal consequence of the aging process, when ovaries gradually become less active and produce less amounts of estrogen and progesterone. Roughly 8 in 10 women experience mild problems or none at all, but some women have severe symptoms. The symptoms include: hot flushes, heavy sweating, anxiety, panic, or depression; drying and wrinkling of the skin, vaginal dryness and discomfort, urinary stress incontinence, cystitis like symptoms, insomnia, irritability, and memory loss.

The current treatment of the above syndromes include hormone replacement therapy (HRT) in the form of pills, implants under the skin and skin patches, applying estrogen creams, combination therapy, calcium tablets, herbal supplements and symptomatic treatment. Out of all the treatments, Estrogen replacement therapy (ERT), aims at replacing the diminish estrogen production, is probably the best known and the most controversial treatment.

However, these measures are not comprehensive and effective in many cases and are prone to a number of side effects including higher incidence of endometrioma, breast and ovary cancer, CHD and stroke.

It is thus an object of the present invention to provide new formulations to treat menopausal syndromes that avoid the above-mentioned side effects and with improved effectiveness.

### SUMMARY OF INVENTION

One promising solution to menopausal syndromes is traditional Chinese herbal medicine, in view of its minimal side effects.

One of the challenges of manufacturing traditional Chinese medicine is that the quality of the herbs from which active ingredients are extracted from varies greatly due to differences in growth conditions, origins, collection time and post-harvest processing. To guarantee that the medicines produced has desired efficacy, it is important to ensure that the herbs used are high and consistent in quality. However, due to the complexity of the ingredients of the herbs, it is difficult and impractical to establish a comprehensive quality control. The current common practice is to select one or two ingredients in an herb as the indicators. The quality of the herb is then assessed by the content of the indicator ingredient in that herb. The drawback of this solution is obvious as the assessment does not tell the content of other ingredients.

It is thus another object of the present invention to provide a method to solve the above-mentioned technical challenges in quality control.

In one aspect of the present invention, a pharmaceutical composition is provided, comprising the extracts of prepared Rehmannia glutinosa Libosch, Epimedium brevicornum Maxim., Cornus officinalis Sieb. et Zucc., Dioscorea opposita Thunb., Lycium barbarum L., and prepared Glycine max (L.) Merr. In a preferred embodiment, the pharmaceutical composition is in the absence of the herbs selected from a group consisting of Poria Cocos (Schw.) Wolf, Paeonia suffruticosa Andr., Cuscuta chinensis Lam., Alisma orientalis (Sam.) Juzep., Codonopsis pilosula (Franch.) Nannf., Bupleurum chinense DC., Glycyrrhiza uralensis Fixch., Pinellia ternate (Thunb.) Breit., Scutellaria baicalensis Georgi, Leonurus japonicus Houtt., prepared Glycyrrhiza uralensis Fixch. or a combination therefrom. In a more preferred embodiment, the Radix Rehmanniae is Shu Di Huang, the Epimedium brevicornum Maxim is Yin Yang Huo, the Cornus officinalis Sieb. et Zucc. is Shan Zhu Yu, the Dioscorea opposita Thunb. is Shan Yao, the Lycium barbarum L. is Gou Qi Zi, and the Glycine max (L.) Merr. is Dan Dou Chi. In a further preferred embodiment, the pharmaceutical composition consist essentially of the extracts of Shu Di Huang, Yin Yang Huo, Shan Zhu Yu, Shan Yao, Gou Qi Zi, and Dan Dou Chi. In an even more preferred embodiment, the pharmaceutical composition consist of the extracts of Shu Di Huang, Yin Yang Huo, Shan Zhu Yu, Shan Yao, Gou Qi Zi, and Dan Dou Chi.

In another preferred embodiment, the extracts are made from the following materials in weight portion:
Shu Di Huang: 4.5-35.5%
Yin Yang Huo: 5.5-24.5%
Shan Zhu Yu: 1.0-25.2%
Shan Yao: 0.5-26.0%
Gou Qi Zi: 3.0-24.0%
Dan Dou Chi: 3.5-22.5%

In a more preferred embodiment, the extracts are made from the following materials in weight portion:
Shu Di Huang: 15-25 portions
Yin Yang Huo: 8-17 portions
Shan Zhu Yu: 5-20 portions
Shan Yao: 10-24 portions
Gou Qi Zi: 10-25 portions
Dan Dou Chi: 9-22 portions

In an even more preferred embodiment, the extracts are made from the following materials in weight portion:
Shu Di Huang: 19.6 portions
Yin Yang Huo: 11.2 portions
Shan Zhu Yu: 10.4 portions
Shan Yao: 14.0 portions
Gou Qi Zi: 14.4 portions
Dan Dou Chi: 15.4 portions

In another aspect of the present invention, a method of preparing the pharmaceutical composition is provided, comprising providing the extracts of Shu Di Huang, Yin Yang Huo, Shan Zhu Yu, Shan Yao, Gou Qi Zi, and Dan Dou Chi.

In a preferred embodiment, the portions of the materials are as follows:
Shu Di Huang: 15-25 portions
Yin Yang Huo: 8-17 portions
Shan Zhu Yu: 5-20 portions
Shan Yao: 10-24 portions
Gou Qi Zi: 10-25 portions
Dan Dou Chi: 9-22 portions

In a more preferred embodiment, the portions of the materials are as follows:
Shu Di Huang: 18-22 portions
Yin Yang Huo: 10-13 portions
Shan Zhu Yu: 8-12 portions
Shan Yao: 12-17 portions
Gou Qi Zi: 13-17 portions
Dan Dou Chi: 14-18 portions

In an even more preferred embodiment, the portions of the herbs are as follows:
Shu Di Huang: 19.6 portions
Yin Yang Huo: 11.2 portions
Shan Zhu Yu: 10.4 portions
Shan Yao: 14.0 portions
Gou Qi Zi: 14.4 portions
Dan Dou Chi: 15.4 portions

In another preferred embodiment, the method further comprises the following steps:
(a) providing a mixture comprising herbs of Shu Di Huang, Yin Yang Huo, Shan Zhu Yu, Shan Yao, Gou Qi Zi, and Dan Dou Chi;
(b) extracting the mixture with water, obtaining a first extract solution;
(c) concentrating the first extract solution, forming a concentrated extract solution;
(d) extract the concentrated extract solution with an ethanol to obtain a second extract solution and precipitation; and
(e) concentrating said second extract solution.

Preferably, step (b) comprises decocting the mixture in water twice; the temperature of the water is preferably at 90-100° C.; the decocting time is preferably 1.5 hours each time.

Preferably, step (c) comprises heating the first extract solution at 80° C.; preferably the solution is concentrated to a relative density of 1.1-1.3.

In a more preferred embodiment, the ethanol is 70-100% (v/v). In yet another preferred embodiment, the concentrating in step (e) comprises concentrating said second extract solution to a relative density of 1.0-1.5.

In another aspect of the present invention, a method of treatment of menopausal syndromes is provided, comprising administration of a therapeutically effective amount of the above-mentioned pharmaceutical compositions.

In yet another aspect of the present invention, it is provided a use of the above-mentioned pharmaceutical compositions in the preparation of a medicament for the treatment of menopausal syndromes.

In still another aspect of the present invention, it is provided a nutritional supplement comprising the pharmaceutical compositions as mentioned above.

In a further aspect of the present invention, a formulation for treating depression is provided, comprising the pharmaceutical compositions as mentioned above, wherein the concentration of the compositions ranges from 1% to 100% by weight.

### BRIEF DESCRIPTION OF THE DRAWINGS

FIG. 1 shows the effects of Composition I on estrogen receptor α (ERα) transactivation in HepG2 cells (n=3, Mean±SD) according to one aspect of the present invention.

FIG. 2 shows the effects of Composition Ion estrogen receptor β (ERβ) transactivation in HepG2 cells (n=3, Mean±SD) according to another aspect of the present invention.

FIG. 3 shows the effects of Composition I on the estradiol-induced ERα transactivation in HepG2. (n=3)

### DETAILED DESCRIPTION

As used in the present specification and claims, the term “comprising” “comprise,” “comprises,” and “comprising” mean “including, but not necessarily limited to”. For example, a method, apparatus, molecule or other item which contains A, B, and C may be accurately said to comprise A and B. Likewise, a method, apparatus, molecule or other item which “comprises A and B” may include any number of additional steps, components, atoms or other items as well. The term “consisting of”, “consisted of”, “consists of” mean that the product (or method) has the recited elements (or steps) and no more. The term “consisting essentially of”, “consisted essentially of”, and “consists essentially of” mean that in addition to the recited elements, the product (or method) necessarily includes the listed elements and is open to unlisted elements that do not materially affect the basic and novel properties of the product (or method). Particularly, “consisting essentially of” excludes other elements from having any essential significance to the combination, that is, it allows some “reading on” additional unspecified substances, i.e., those which do not materially affect the basic and novel characteristics of the claimed invention.

As used herein, the expression “the composition is in the absence of the herbs selected from a group consisting of A, B, C, D” refers to a composition that does not contain either A, B, C, D, any kinds of combinations thereof’.

As used herein, the terms “herb” and “herbs” refer to the plant-based materials with therapeutic effects.

As used herein, the terms “Shu Di Huang”, “Yin Yang Huo”, “Shan Zhu Yu”, “Shan Yao”, “Gou Qi Zi”, and “Dan Dou Chi” refer to the traditional Chinese herbs defined below. For ease of references, the naming of the above herbs is based on the official pronunciation system of the People's Republic of China. Detailed information regarding the herbs could be referred in “Pharmacopoeia of People's Republic of China” (2000 edition, Part 1).

Shu Di Huang, also known as Shu Di, Prepared Rehmannia Root, Radix Rehmanniae Preparata, is the processed Radix Rehmanniae.

Yin Yang Huo, also known as Epimedium, Epimedium Leaf, Herba Epimedii (Latin), is the dried leaf of Epimedium sagittatum (Siebold. & Zucc.) Maxim. It belongs to the barberry family (Berberidaceae). Native to east Asia, the plant is grown on hillsides in damp shady bamboo groves or in moist woodlands in China and Japan.

Shan Zhu Yu, also known as Asiatic Cornelian Cherry Fruit, Fructus Corni, is the dried ripe sarcocarp of Cornus officinalis Sieb. et Zucc. It belongs to the Cornaceae family. It is sour and astringent in taste and slightly warm in nature.

Shan Yao, also known as Chinese Yam, Rhizoma Dioscoreae (Latin), Cinnamon Vine and Cinnamon Yam, is the root of the perennial herbaceous trailing plant Dioscorea opposita (Thunb.). It belongs to the Diosoreaceae family. The plant is widely cultivated in temperate east Asia, and can be found in valleys and on the sunny slopes of hills in China, India and Japan.

Gou Qi Zi, also known as Wolfberry fruit, Fructus Lycii (Latin), is the dried mature fruit of the plant Lycium barbarum L. and belongs to the Solanaceae family. The fruits are the berries of Lycium chinense and more commonly Lycium barbarum. The roots also have healing properties. Native to east Asia and Europe, it grows wild on hillsides in the cooler regions of northern China and Europe. However, it is also grown as a cultivated plant in almost all parts of China and in some other regions of Asia, as well as naturalized in Britain, the Middle East and North America.

Dan Dou Chi, also known as Tasteless Fermented Soybean or black soybean, is the ripe seed of Glycine max (L.) Merr. It belongs to the family Leguminosae, which are processed by press steaming and later fermented. As a condiment, salted fermented black soybean is used; as an herb, the taste less fermented black soybean is used.

As used herein, the term “decoction” refers to boiling in water at a temperature between 90-120° C., preferably 90-100° C., The effective ingredients were extracted from the herbs in such boiling water.

The experiments are divided into three sections. The first section illustrates an example of how the composition of the present invention is prepared. The second section describes experiments conducted to test the efficacies of the Composition. The tests in the second section comprise animal studies and in vitro studies. The animal studies include the acute toxicity study, the sleeping test, the open field test, the dark-light box tests on normal and ovariectomized mice, the elevated plus-maze test on ovariectomized rats, the step-down test, and the memory test. The in vitro tests were targeted to test the composition's effectiveness in regulating serum estrogen and progesterone levels, anti-oxidation, preventing cardiovascular diseases, the ability to induce the transcriptional activation of ERα and ERβ in HepG2 cells and its influence on estradiol-induced ER transactivation.

Principles, materials used, and procedures of each test are detailed in the following examples.

Section I. Preparing the Composition

### EXAMPLE 1

### Preparation the Composition

Materials:

Shu Di Huang: purchased from Shanghai Hua Yu Pharmaceutics Co., Ltd.
Yin Yang Huo: purchased from Shanghai Hua Yu Pharmaceutics Co., Ltd.
Shan Zhu Yu: purchased from Shanghai Hua Yu Pharmaceutics Co., Ltd.
Shan Yao: purchased from Shanghai Hua Yu Pharmaceutics Co., Ltd.
Gou Qi Zi: purchased from Shanghai Hua Yu Pharmaceutics Co., Ltd.
Dan Dou Chi: purchased from Shanghai Hua Yu Pharmaceutics Co., Ltd.
95% ethanol: purchased from Shanghai Lai Yin Chemicals Co., Ltd., product code: 030801
Starch: purchased from Shanghai Hua Yu Pharmaceutics Co., Ltd., product code: F20020812

A total of 85 kg of 19.6 kg Shu Di Huang, 11.2 kg Yin Yang Huo sliced to 5 mm in width, 10.4 kg Shan Zhu Yu, 14.0 kg Shan Yao sliced to 3 mm in thickness, 14.4 kg Go Qi Zi and 15.4 kg smashed Dan Dou Chi in 20 mesh were decocted in eight times (w/v) of boiling water twice, 1.5 hours each time. The two batches of decocted liquid were combined, filtered and concentrated at 80 oC to a relative density of 1.2. Twice the volume of 95% ethanol was used to precipitate impurities. The supernatant was concentrated at 50-80 oC to a relative density of 1.3. The composition mixture was dried at 70 oC and then crushed into powder, filtered with 20 mesh and finally got the powder (Composition I, 10.5 Kg). Each gram of Composition I contained approximately 8.10 g crude drug (crude herb).

Section II. Testing the Properties and Efficacies of Composition I

### EXAMPLE 2

### Acute Toxicity Study

The acute toxicity study was carried out as described in Instruction of Toxic Research of Chemical Medicine Administration and Xu S Y. (1991 ).

Animals:

20 KM mice, 10 male and 10 female, each weighed between 18-22 g, purchased from Shanghai Experimental Animal Center, the Chinese Academy of Sciences.

Drug:

Composition I as prepared in Example 1.

Method:

The formulation was prepared on the day of testing by dissolving 5 g of Composition I in 20 ml of saline water. The mice were fasted for five hours before testing. At the dose of 5 g/kg, the formulation was administrated intragastrically (i.g.) to the mice. After administration, the mice were examined continuously for 10 days for their physical and mental behavior (observed in the following study). After 10 days, all major organs of the mice were dissected and observed.

Result:

The LD50 (half lethal dose) of Composition I was 497 g (crude herb)/kg for female mice and 528 g (crude herb)/kg for male mice, respectively.

### EXAMPLE 3

### Sleeping Test

According to Xu S Y. (1991 ), Chen Q. (1996 ), Zhang J T. (1998 ), Zhu X D. and Tang X C. (1998 ), an agent is deemed to have sedative and hypnotic effects if it can prolong the duration of the sleep, as indicated by the disappearance of right reflex, of pentobarbital sodium-injected mice

Animals:

KM mice, females, weighed between 18-20 g, purchased from Shanghai Experimental Animal Center, the Chinese Academy of Sciences.

Drug, dose, and the administration formulation:
For negative control group: 0.9% saline.
For positive control group: Diazepam, purchased from BangNing Pharmaceutical Co. Ltd. The dose was 0.5 mg/kg weight/d. The administration formulation was formed by dissolving 1 mg of Diazepam in 40 ml of water.
For the test group: Composition I as prepared in Experiment 1. The dose was 56 g (crude herb)/kg weight/d. The administration formulation was formed by dissolving 0.56 g of Composition I in 0.2 ml of water.
   Method:

Thirty normal female mice weighed between 18-20 g were randomized into 3 groups of 10: the negative control group, the positive control group, and the test group. For the negative control and testing group, the drugs were administered to the mice orally with the doses and formulations indicated, once per day for consecutive 14 days. For the positive control group, the drug was given on the day fourteen orally. One hour after the last administrations, all mice were injected with 35 mg/kg of pentobarbital sodium intraperitoneally. The duration of the disappearance of righting reflex was observed and recorded. According to Zhang J T. (1998 ), the duration of the disappearance of righting reflex equals to the sleeping time.

Result:

Table 1 shows the effectiveness of the drugs in prolonging the sleeping time of the pentobarbital sodium-injected mice.

TABLE 1

| Group | Sleeping Time (sec) |
|---|---|
| Negative control group | 1510.7 ± 898.2 |
| Positive control group | 2927.8 ± 1061.2** |
| The test group | 2679.7 ± 1045.6* |

*p < 0.05,
**p < 0.01

The test group showed a significant effect in prolonging mice sleeping times.

### EXAMPLE 4

### Open Field Test

According to Xu S Y. (1991 ) and Chen Q. (1996 ), an agent is deemed to be effective in suppressing the central nervous system (CNS) or have seductive effects if it can reduce the spontaneous activity in animals.

Animals:

KM mice, females, weighed between 18-20 g, purchased from Shanghai Experimental Animal Center, the Chinese Academy of Sciences.

Materials:

Multi-functional Auto-Motion Recorder for Mouse/Rat: manufactured by Facility Station of Shandong Academy of Medical Science. Product code: YLS-1B.

Drug, dose, and the administration formulation:
For negative control group: 0.9% saline.
For positive control group: Diazepam as described above. The dose was 1.0 mg/kg weight/d. The administration formulation was formed by dissolving 1 mg of Diazepam in 20 ml of water.
For the test group: Composition I as prepared in Experiment 1. The dose was 14 g (crude herb)/kg weight/d. The administration formulation was formed by dissolving 0.14 g of Composition I in 0.2 ml of water.
   Method:

Thirty normal female mice weighed between 18-20 g were randomized into 3 groups of 10: negative control, positive control and the test group. For the negative control and testing group, the drugs were administered to the mice orally with the doses and formulations described above, once per day for consecutive 14 days. For the positive control group, the drug was given on the day fourteen orally. One hour after the last administration, the mice were individually placed into a box with infrared detection light to record their spontaneous activities in 3 minutes. The average locomotion times of the mice in each group during the period were recorded and summarized in Table 2.

TABLE 2

Locomotion times
during the 3 minutes

| Group | 1 hour after drug | 3 hour after drug |
|---|---|---|
| Negative control | 74.3 ± 8.5 | 56.0 ± 10.0 |
| group | | |
| Positive control | 61.0 ± 11.6* | 46.2 ± 11.3* |
| group | | |
| The test group | 65.6 ± 8.5* | 37.9 ± 8.43** |

*p < 0.05,
**p < 0.01

The result showed that Composition I has a significant effect (p<0.05 ) in reducing the spontaneous activities of animals; the effect is comparable to that of the positive control group. The effectiveness of Composition I persists 3 hours after administration.

### EXAMPLE 5

### Dark-Light Box Test on Normal Mice

In the experiment, mice were placed in Shimada light and dark box. The times of mice shuttling between the boxes and the duration of mice staying in the dark/light box were recorded. According to Xu S Y (1991 ), Chen Q. (1996 ), Zhang J T. (1998 ), agents that increase the shuttle times and shorten the duration of mice staying in the dark boxes are deemed effective in treating anxiety.

Apparatus:

Shimada light and dark box: size: 40 cm×40 cm×12 cm, having a light box (20 cm×20 cm×12 cm) at the center, and two dark boxes (10 cm×10 cm×12 cm) at two diagonal corners. Two L-shaped tracks are connected to two sides of each dark box. Manufacturer: Shanghai Institute of Pharmaceutical Industry.
   Animals:

KM mice, female, 18-20 g, purchased from Shanghai Experimental Animal Center, the Chinese Academy of Sciences.

Drug and Dose:

Negative control group: 0.9% saline, 0.2 ml/10 g weight/d
Positive control group: Diazepam, 1.0 mg/kg weight
Test group: Composition I, 56 g (crude herb)/kg weight/d
   Method:

Thirty normal female mice weighed between 18-20 g were randomized into 3 groups of 10: negative control, positive control (Diazepam) and the test group. The mice in the negative and test groups were administered orally with respective drugs and doses as indicated above, once a day for consecutive 14 days. The mice in the positive control group received only one dose orally as indicated above on day fourteen. 1 hour after the last administration on day fourteen, all the mice were placed in the Shimada light and dark box. The shuttle times and duration of mice staying in the dark box within 10 minutes were recorded and summarized in Table 3.

TABLE 3

| | Behaviors of mice in | |
|---|---|---|
| | Shimada Dark-Light Box | |
| Group | Shuttle Time | Time spent in dark box |
| Negative control group | 60.8 ± 8.46 | 247.9 ± 58.75 |
| Positive control group | 76.1 ± 8.90** | 166.6 ± 45.05** |
| The test group | 72.8 ± 7.91** | 169.8 ± 61.22** |

**p < 0.01

The result showed that Composition I has a significant effect (p<0.01) in increasing the shuttle times and (p<0.01) shorten the duration of mice staying in the dark box compared with the negative control group.

### EXAMPLE 6

### Dark-Light Box Test on Ovariectomized Mice

The same experiment of Example 5 was conducted on ovariectomized mice. Agents that are able to increase the shuttle times of mice and shorten the duration of staying in the dark boxes are believed to be effective in treating anxiety. See Xu S Y. (1991), Chen Q. (1996), Zhang J T. (1998).

Apparatus: Same as Example 5.

Animals: Same as Example 5.

Drug and Dose:

Negative control group: 0.9% saline,

Positive control group: Diazepam, 1.0 mg/kg weight

Test group: Composition I, 14 g (crude herb)/kg weight/d

Method:

Thirty female mice were anaesthetized by intraperitoneal injection with 35 mg/kg Pentobarbital Sodium. Two cuts were made on the back of the rats, each cut being 1.5 cm from the spine. The two ovaries were taken from the two cuts, respectively. The cuts were then sealed. 1 week after the ovariectomization, the 30 mice were randomly divided into 3 groups, and the same administration procedures as Example 5 were followed, using drugs and doses specified above. 1 hour after the last administration on day fourteen, all the mice were placed in the Shimada light and dark box. The shuttle times and duration of mice staying in the dark box within 10 minutes were recorded and summarized in Table 4.

TABLE 4

Transition Time in
Shimada Dark-Light Box

| Group | Shuttle Time | Time spent in dark box |
|---|---|---|
| Negative control | 21.2 ± 8.6 | 92.2 ± 39.3 |
| group | | |
| Positive control | 20.5 ± 10.7 (p = 0.874) | 47.4 ± 31.9* |
| group | | |
| The test group | 8.0 ± 6.4** | 48.1 ± 39.7* |

*p < 0.05,
**p < 0.01

The result showed that Composition I has a significant central suppressive effect (See Xu S Y. (1991)) and could shorten the duration of staying in the dark box of ovariectomized mice.

### EXAMPLE 7

### Elevated Plus-Maze Test on Ovariectomized Rats

The elevated plus maze is a popular paradigm for the study of anxiety in rats. The apparatus generally consists of two open arms and two closed arms, with the four arms intersecting to form the shape of a plus sign; the whole apparatus is elevated from the floor. The combination of height and open space is assumed to induce anxiety-related behavior (i.e. avoidance of the open arms). According to the Xu S Y (1991), Chen Q (1996), Zhang J T. (1998), agents that increase the times of rats entering into the open arms and duration of staying in the arms of the maze are believed to be effective in treating anxiety.

Animals:

SD rats, female, weighed 230-250 g, purchased from Shanghai Experimental Animal Center, the Chinese Academy of Sciences.

Drugs and Doses:

For negative control group: 0.9% saline.
For positive control group: Diazepam as described above. The dose was 0.5 mg/kg weight.
For test group: Composition as prepared in Experiment 1. The dose was 14 g (crude herb)/kg weight/d.
   Method:

Thirty female rats were divided into the above-mentioned 3 groups randomly and were anaesthetized by intraperitoneal injection with 60 mg/kg Pentobarbital Sodium. Two cuts were made on the back of the rats, each cut being 1.5 cm from the spine. The two ovaries were taken from the two cuts, respectively. The cuts were then sealed. The drug administration began 1 week after the ovariectomization. The rats in the negative and test groups were administered orally with saline and Composition I at doses indicated above, once a day for consecutive 14 days. The rats in the positive control group received Diazepam orally only once on day fourteen. 1 hour after the last administration, the rats were placed in a maze. The times of rats entering the open arms and the duration of staying in the open arms were recorded and summarized in Table 5. See Xu S Y (1991) and Zhang J T. (1998).

TABLE 5

| | Transition Time in Maze | |
|---|---|---|
| | Times of entering | Time spent in |
| | open arm × 100% | open arm × 100% |
| | Times of entering | Time spent |
| Group | both arms | in both arms |
| Negative control | 2 ± 4% | 0.5 ± 1% |
| group | | |
| Positive control group | 12 ± 11%* | 6 ± 7%* |
| The test group | 11 ± 17% (p = 0.3) | 4 ± 5% (p = 0.48) |

*p < 0.05

The results showed that Composition I increased the times of ovariectomized rats entering the open arms and the duration of ovariectomized rats staying in the open arms.

The results of the Experiments 5-7 showed that Composition I is effective in treating anxiety.

### EXAMPLE 8

### Step-Down Test on Normal Mice

A step-down recorder, comprising a platform and a copper floor, is provided. The mice were placed on the copper floor. When the cooper floor is electrified, the normal reaction of the mice is to jump up to the platform to avoid the electric shock. After a period of time, the mice may forget about the shock and jump back to the floor. This jumping back to the floor is an “error reaction”. The mice that jump back to the floor received electric shock again and would jump back to the platform again. The duration between the first jump up (to the platform) and back (to the floor) is called the “latency period”. The number of jumping backs (error reactions) in a certain period of time could serve as an indicator of the memory of the mice.

Based on the Principle, agents that are able to reduce the error reactions of mice in a certain period of time and shorten the latent period are deemed effective in memory improvement. Xu S Y. (1991), Chen Q. (1996), Zhang J T. (1998),Yang J, Wang J, et al. (2000).

Animals: KM mice, female, weighed 18-20 g, purchased from Shanghai Experimental Animal Center, the Chinese Academy of Sciences.

Drug and doses:

Scopolamine: purchased from Shanghai Hefeng Pharmaceutical Ltd. Dose: 2 mg/kg weight
The negative control group: 0.9% saline
The positive control group: Huperzine A, purchased from Shanghai Fudan Fuhua Pharmaceutical Ltd., at the dose of 1.5 ug/kg weight/d (equal to 0.2 ml/10 g weight)
The test group: Composition I, at the dose of 14 g (crude herb)/kg weight/d
   Apparatus:
Step down recorder. Code: YLS-2 T, purchased from Facility station of Shandong Academy of Medical Science
   Method:

Thirty normal female mice were randomized into 3 groups of 10: negative control, positive control (Huperzine A) and the test group. Mice in the three groups received respective drugs and doses orally as indicated above, once per day for consecutive 14 days. An hour after the last administration, all the mice were administrated with 2 mg/kg scopolamine intraperitoneally. 30 minutes later, the mice were placed in the step down recorder for 5 minutes for pre-adaptation. After the pre-adaptation, the copper floor was electrified for 5 minutes. The number of electric shocks each mouse received in the 5 minutes was recorded. After 24 hours, the test was conducted again.

The number of shocked mice, the latency period of each mouse, and the average number of error reactions in 3 minutes were recorded. Results were summarized in Table 6.

TABLE 6

| | 5 minutes | | 24 hours after | | |
|---|---|---|---|---|---|
| | pre-adaptation | | scopolamine administration | | |
| | Total | Number of | Number of | Number of error | First step |
| | number | shocks per | shocked | reactions in | down latency |
| Group | of shocks | mouse | mice | 3 minutes | period (sec) |
| Negative | 41 | 4.1 ± 1.9 | 9 | 14 | 77.1 ± 94.9 |
| control | | | | | |
| Positive | 51 | 5.7 ± 2.7 | 5 | 8 | 200.6 ± 137.2* |
| control | | | | | |
| (Huperzine A) | | | | | |
| The test group | 28 | 3.1 ± 1.9 | 4 | 5 | 181.3 ± 141.7 |
| | | | | | (p = 0.074) |

*p < 0.05 compared to the negative control group (n = 10, mean ± SD)

The results showed that Composition I has an effect in prolonging the step down latency period in shocked mice, but not at the level of statistical significance.

### EXAMPLE 9

### Step Through Test on Normal Mice

A Step Through Test Box for Mouse with a light room and a dark room inside is provided. The mice are placed in the light room with their back facing the door. When the mice come out from the light room and enter the dark room, they get shocked, such behavior is called an error reaction. The first duration before an error reaction occurs after a mouse is placed in the box is called the latency period. Mice bearing better memories have longer latency period and commit less error reactions in a certain period of time.

Agents that are able to increase the latency period and reduce error reactions of mice in the Step Through Test Box are deemed effective in improving the memory of animals. Reference: Xu S Y (1991), Chen Q (1996), Zhang J T. (1998), Yang J, Want J. et al (2000), and Zhu X D and Tang X C. (1998), page 492-497.

Animals: KM mice, female, 18-20 g, purchased from Shanghai Experimental Animal Center of the Chinese Academy of Sciences.

Drug and Doses:

45% Ethanol: purchased from China Medicine Group Shanghai Chemical Reagent Corporation
The negative control: 0.9% saline
The positive control: Huperzine A, purchased from Shanghai Fudan Fuhua Pharmaceutical Ltd., at the dose of 1.5 ug/kg weight/day (0.2 ml/10 g weight).
The test group: Composition I, at the dose of 14 g (crude herb)/kg weight/day
   Apparatus:
Step Through Test Box for Mouse: manufactured by Facility station of Shandong Academy of Medical Science; product code: YLS-1A
   Method:

Thirty normal female mice weighed between 18-20 g were randomized into 3 groups of 10: negative control, positive control (Huperzine A) and the test group. The mice were administered orally with the above-indicated doses and drugs, once a day for 14 days. An hour after the last administration (day 14), all the mice were orally given 45% ethanol (0.1 ml/10 g). 30 minutes later, the mice were placed in the light room of the test box with their back facing the door. When the mice entered the dark room and got shocked, the latency period was recorded (the 1^{st} latency period). The test was conducted again 24 hours later, wherein the number of mice entering the dark room (error reaction) and the latency period (the 2^{nd} latency period) were recorded. The results were summarized in Table 7.

TABLE 7

| | The first test | The second test (24 h later) | |
|---|---|---|---|
| | The 1^{st} latency | No. of error | The 2^{nd} latency |
| Group | period | reactions | period |
| Negative control | 16.8 ± 10.21 | 4.7 ± 3.8 | 82.4 ± 75.3 |
| Positive control | 19.3 ± 16.16* | 1.4 ± 2.0* | 196.2 ± 121.9* |
| (Huperzine A) | | | |
| The test group | 24.8 ± 22.43 | 2.7 ± 2.6 | 143.9 ± 110.4 |
| | (p = 0.75) | (p = 0.17) | (p = 0.196) |

*p < 0.05, compared with the negative control group

The results showed that Composition I has an effect in prolonging the latency period of ethanol-induced memory impairment, but no statistical differences were detected.

### EXAMPLE 10

### The Effect on Regulating Serum Estrogen and Progesterone Levels in Ovariectomized Rats

Female hormone stimulates estrogen in overiectomized female mice/rats.

Pharmacy Pharmacology Toxicology (1996); 60-61, Xu S Y, et al, (2003), Gu Z P, et al. (1990), W. G (2001).

Animals:

SD rats, female, weighted between 220-250 g before administration, purchased from Shanghai Sino-British Sippr/BK LAB Animal Ltd., randomly split into 5 groups.

Drug:

Negative control group: 0.9% saline
Positive control group:
   1. Estradiol Benzoate, purchased from Shanghai No. 9 Pharmaceutical Ltd. Dose: 0.01 mg/kg weight/day
   2. ZuoGuiWan, purchased from Shanghai Leiyunshang Fengbang Pharmaceutical Co., Ltd.
   3. GengNianAn, purchased from Zhongxin Pharmacy Tianjin Leren Tang Pharmaceutical Factory. Dose: 6 g/kg weight
The test group: Composition I
0.5% Sodium salt of carboxymethylcellulose (CMC—Na): manufacturer: China Medicine Group Shanghai Chemical Reagent Corporation
   Formulation and Doses:
Negative control group: 0.5% CMC—Na. Dose: 1.0 ml/100 g weight/day
Positive control group:
   1. Estradiol Benzoate, Dose: 0.10 ml/10 weight/day dissolved in peanut oil (0.01 mg/ml).
   2. ZuoGuiWan, Dose: 7 g/kg weight/day dissolved in 0.5% CMC—Na,
   3. GengNianAn, Dose: 6 g/kg weight/day dissolved in 0.5% CMC—Na,
5. The test group: Composition I, dose7 g (crude herb)/kg weight/day
0.5% Sodium salt of carboxymethylcellulose (CMC—Na): manufacturer: China Medicine Group Shanghai Chemical Reagent Corporation
   Method:

Thirty ovariectomized female SD rats weighed between 220.0-250.0 g were randomized into 5 groups: the negative control group, the positive control group, comprising 1. Estradiol Benzoate group, 2. ZuoGuiWan group, 3. GengNianAn group, and the test group. The rats in the negative control, ZuoGuiWan, GengNianAn and the test groups received respective drug and dose as indicated intragastrically. The rats in Estradiol Benzoate were injected with estradiol at the above-indicated dose. All rats received administration once everyday for consecutive 28 days. During the 28 days, a daily pap smear was performed before drug administration. Twenty-four hours after the last administration, all of the rats were weighed, anesthetized, blood withdrawn and killed. The uterus was then removed and half of the uterus was weight in wet. The other half of the uterus was dried for 8 hours in an oven for the measurement of dried weight. The serum estrogen and progesterone levels were determined using radioimmunoassay (RIA) method. The serum estrogen and progesterone levels of ovariectomized rats were summarized in Table 8. The uterus weight (wet) and visceral index of ovariectomized rats were summarized in Table 9.

TABLE 8

| | | | Serum |
|---|---|---|---|
| | | | Progesterone |
| Group | | Serum Estrogen (ng/ml) | (ng/ml) |
| Negative control | | 15.67 ± 5.01 | 25.75 ± 4.71 |
| Positive | Estradiol Benzoate | 33.00 ± 21.76** | 25.85 ± 6.15 |
| control | | | (p = 0.974) |
| | ZuoGuiWan | 23.40 ± 12.58 | 20.59 ± 2.94 |
| | | (p = 0.198) | (p = 0.063) |
| | GengNianAn | 24.17 ± 5.35* | 20.21 ± 3.50* |
| The test | Composition I | 26.00 ± 12.52 | 14.35 ± 4.06* |
| group | | (p = 0.09) | |
| | Composition II | 20.83 ± 8.89 | 18.80 ± 5.44* |
| | Composition III | 13.50 ± 6.54 | 20.71 ± 9.22 |

mean ± SD, (n = 6),
*p < 0.05,
**p < 0.01

TABLE 9

| | | Wet weight of uterus | Visceral Index |
|---|---|---|---|
| Group | | (g) | (g/100 g BW) |
| Negative control | | 0.090 ± 0.017 | 0.034 ± 0.007 |
| Positive | Estradio | 0.597 ± 0.064** | 0.246 ± 0.030** |
| control | ZuoGuiWan | 0.122 ± 0.050** | 0.045 ± 0.019* |
| | GengNianAn | 0.124 ± 0.028 | 0.046 ± 0.010 |
| | | (p = 0.077) | (p = 0.056) |
| The test | Composition I | 0.121 ± 0.010* | 0.073 ± 0.007* |
| group | Composition II | 0.137 ± 0.036* | 0.050 ± 0.013* |
| | Composition III | 0.149 ± 0.070 | 0.060 ± 0.037 |

mean ± SD, (n = 6),
*p < 0.05,
**p < 0.01

The results in Table 8 showed that the compositions in the test group elevated both the serum estrogen and progesterone levels, with the progesterone level has statistical significance. The results in Table 9 showed that the compositions in the test group enhanced both the uterus weight and visceral index of ovariectomized rats, having statistical significance compared with the negative control group. The effect is better than the ZuoGuiWan group and the GengNianAn group.

The above observations shown that the compositions I, II, and III, when compared with the negative control, had a significant effect in the uterus weight, visceral index, serum estrogen and progesterone levels. The result is better than those of ZuiGuiWan and GengNianAn. Thus this formulation can also be used in regulating serum estrogen and progesterone levels.

### EXAMPLE 11

### Anti-Oxidation Effect of Composition I—Effects on Copper II Ions-Mediated LDL Oxidation

A substance that prevents the oxidation of low density lipoprotein (LDL) has anti-oxidation effect. In the experiment copper ions were added to induce oxidation of LDL. By examining the levels of LDL oxidation at different time intervals, the anti-oxidation effect of a substance can be measured.

See Puhl H. Waege G and Esterbauer H. (1994), Buege Ja. and Aust S D. (1978).

Drug:

The negative control group: 0.9% saline water
The positive control group: Estradiol Benzoate, purchased from Innovative Research of America, Florida.

The test group: Composition I was dissolved in water at concentrations listed in Table 10A

TABLE 10A
Concentrations of Composition I (mg/ml)
0.01
0.05
0.10

50 μM CuSO₄: purchased from Riedel-deHaen; product code: 31294; catalogue no. 2811; preparation method: 16 mg CuSO₄ added to 100 ml H₂O, 5 ml of this solution added to 95 ml H₂O to obtain 100 ml of 50 μM CuSO₄ solution
LDL: prepared in-house. To prevent lipoprotein modification, EDTA and NaN₃ were added to the serum collected from healthy subjects at the Prince of Wales Hospital. The final concentrations of EDTA and NaN₃ were 0.1% and 0.05% respectively. LDL was isolated from the serum according to the method described previously (Havel et al., 1995). In order to prevent LDL from oxidation, the serum were flushed with nitrogen gas, centrifugated at 1,500 Rotation per minute (rpm) for 15 mins to remove cells and cell debris.
NaCl-KBr solution (153 g NaCl, 354 g KBr and 100 mg EDTA were dissolved in 1L water, density 1.33 g/mL) was then added to increase the density to 1.019 g/ml. The serum was re-centrifugated at 160,000 rpm for 20 hours at 4° C. After the removal of the top layer containing chylomicron and very low-density lipoprotein (VLDL), the density of the remaining serum layer was increased to 1.064 g/mL and was re-centrifugated at 160,000 rpm for 24 h at 4° C. The top LDL layer was collected and flushed with nitrogen and stored at −70° C. until use.
EDTA, Sigma
TBA-TCA-HCL solution: prepared by mixing 0.6 g TBA, 0.0268 g TCA and 0.1 mole of HCl.
thiobarbituric acid, Sigma
Malonaldehyde, Sigma
Tetramethoxypropane, Sigma
   Apparatus:
UV-visible spectrophotometer: manufacturer: Shimadzu, product code UV-1601
   Method:

Four tubes representing the negative control (1 tube) and the test group (3 tubes) were prepared in triplicate. Each tube contained 0.4 μL LDL and was added with 50 μl of 50 μM CuSO₄, respectively. In the 4 tubes of the test group, 50 μL of Composition I at different concentrations as indicated in Table 10A was added, respectively. In the tube of the negative control group, 50 μL of water was added. All of the tubes were incubated at 37° C. At different time intervals, the reaction was quenched by cooling the entire contents of each tube to 4° C. and adding 25 μl of EDTA. After the reaction was quenched, 2 ml of TBA-TCA-HCL solution was added to each sample. The content of LDL in each sample was observed under 532 nm. The levels of the LDL oxidation at different time intervals in the samples containing Composition I at different concentrations are summarized in Table 10B.

TABLE 10B

| | Concentration of Composition I (mg/ml) | | | |
|---|---|---|---|---|
| | 0 (the | | | |
| | negative | | | |
| Incubation | control | | | |
| Time (min) | group) | 0.01 | 0.05 | 0.10 |
| 0 | 11.29 ± 0.99 | 10.76 ± 0.31 | 11.59 ± 0.89 | 12.95 ± 0.84 |
| 120 | 31.38 ± 0.99 | 8.39 ± 0.72 | 14.43 ± 0.84 | 12.36 ± 0.36 |
| 240 | 89.35 ± 1.13 | 34.34 ± 1.26 | 19.41 ± 1.87 | 15.73 ± 2.86 |
| 360 | 88.04 ± 4.07 | 90.36 ± 1.68 | 23.30 ± 3.28 | 19.64 ± 1.52 |
| 480 | 87.33 ± 1.17 | 88.94 ± 1.68 | 69.60 ± 4.54 | 26.64 ± 2.24 |
| 600 | 85.72 ± 2.43 | 88.72 ± 1.68 | 93.78 ± 2.62 | 30.35 ± 5.36 |
| 720 | 83.72 ± 2.43 | 87.72 ± 1.68 | 93.78 ± 2.62 | 30.16 ± 0.81 |
| 840 | 83.72 ± 2.43 | 86.72 ± 1.68 | 93.78 ± 2.62 | 32.74 ± 0.57 |
| 960 | 83.72 ± 2.43 | 85.72 ± 2.68 | 98.78 ± 2.62 | 33.27 ± 0.54 |
| 1080 | 82.72 ± 3.43 | 87.72 ± 1.68 | 98.78 ± 2.62 | 34.87 ± 3.75 |
| 1200 | 83.72 ± 2.43 | 85.72 ± 1.98 | 98.78 ± 2.62 | 69.30 ± 3.26 |
| 1320 | 81.72 ± 2.93 | 86.72 ± 1.68 | 98.78 ± 2.62 | 92.89 ± 1.92 |

n = 3, mean ± SD

The data presents the TBARS values (nmol MDA/μg LDL protein), which is a pink pigment with an absorbance of 532 nm. The absorbance is proportional to the degree of LDL oxidation.

The data in Table 10B showed that the LDL in the control group was rapidly oxidized, while that in the test groups containing Composition I were oxidized much slowly. At the concentration of 0.01 mg/ml, the oxidation can be delayed by 120 minutes. At 0.05 mg/ml, oxidation is delayed by 360 minutes. At 0.10 mg/ml, the oxidation was delayed by 1080 minutes. The above data showed that Composition I has obvious anti-oxidation effects.

### EXAMPLE 12

### Anti-Oxidation Effect of Composition I—Test on the Reduction of Fe³⁺ to Fe²⁺

The higher the anti-oxidation level, the more Fe²⁺ ions are reduced from Fe³⁺ ions. See Benzie I F and Szeto Y T (1999).

Animals:

47 female hamsters, weighed about 100 g, bred at the Laboratory Animal Services Center (LASEC) of the Chinese University of Hong Kong, were randomly divided into 6 groups.

Drug and reagent:

The test groups: Composition I at different doses (Group 1): 16.6 mg/100 g/day, (Group 2): 33.2 mg/100 g/day, and (Group 3): 66.4 mg/100 g/day.
The positive control group (Group 4):_{—}17β Estradiol, purchased from Innovative Research of America, Florida.
The negative control group (Group 5): water
The sham operated group (Group 6): hamsters received the operation but not ovariectomy.
FRAP reagent: prepared by mixing 25 μl 10 mM TPTZ, 25 μl 20 mM FeCl₃6H₂O and 250 μl 300 mM acetate buffer.
   Apparatus:
UV-visible spectrophometer: manufacturer Shimadzu, product code: UV-1601

Method: 47 female hamsters weighed 100 g were divided into 6 groups, including three test groups 1, 2, and 3, the positive control group 4, the negative control group 5, and the sham operated group 6.

The hamsters in groups 1-5 were ovariectomized. A month afterwards, the hamsters in groups 1-6 were administered intragastrically with the drugs and doses indicated above, once every day for consecutive 4 weeks. After 4 weeks of administration, blood samples were obtained from the orbital venous sinus, and were subjected to centrifugation at 10,000 rpm for 10 minutes to collect serum. 10 μl of serum was diluted with 490 μl double distilled water. 300 μl freshly prepared FRAP reagent was then added to initiate the reaction. The samples were placed under 593 nm to observe the changes of absorbance during the first 10 minutes of the reduction of Fe³⁺ to Fe²⁺. The increase in absorbance is an indicator of the level of reduction. The Ferric Reducing Ability of Plasma (FRAP) values were determined as follows:

Calculation:
FRAP value (nmol/L)=[standard solution ×ΔA_{10-0 mins}]/[sample of ΔA_{10-0 mins}]

The percentage of FRAP value remained after 4 weeks of extract treatment=FRAP value after 4 weeks of administration/FRAP value before administration]×100%.

TABLE 11

| | Percentage of FRAP value | |
|---|---|---|
| | remained after 4 weeks of | Number of |
| Group | treatment⁺ | hamsters |
| 1. Low dosage (16.6 mg/ | 92.85 ± 28.68 (p = 0.16) | 8 |
| 100 g/day) | | |
| 2. Medium dosage | 96.43 ± 20.27* (p = 0.03) | 7 |
| (33.2 mg/100 g/day) | | |
| 3. High dosage | 90.17 ± 30.75 (p = 0.25) | 8 |
| (66.4 mg/100 g/day) | | |
| 4. Positive control | 88.32 ± 21.06 (p = 0.17) | 8 |
| 5. Negative control | 75.72 ± 13.04 | 9 |
| 6. Sham-operated | 123.23 ± 67.23** (p = 0.001) | 7 |

(n = 7-9, mean ± SD)
*p < 0.05 when compared to negative control;
**p < 0.01 when compared to negative control

The FRAP value of the test group (medium dosage) was significantly higher than that of the negative control group, thereby indicating that Composition I can significantly slow down the oxidation process.

### EXAMPLE 13

### Cardiovascular Diseases Preventing Effects—Changes in Serum[Non-HDL]/[HDL]Ratio and Kidney Cholesterol

Serum non-HDL/HDL ratio is served as an indicator for the incidence of cardiovascular diseases. The total cholesterol level in different organs exerts positive co-relationship with cardiovascular diseases.

See Chan P T, Fong W P, Cheung Y L., Huang Y, H O K K W. and Chen Z Y (1999).

Animals: hamsters, female, weighed 100 g, bred at the Laboratory Animal Services Center (LASEC) of the Chinese University of Hong Kong, randomly divided into 6 groups.

Group/Drug/Dose:

The negative control group: 0.9% saline
The test group: administered with Composition I at doses 16.6 mg/100 g/day, 33.2 mg/100 g/day, and 66.4 mg/100 g/day.
The positive control group: 17β Estradiol 21 day release, purchased from Innovative Research of America, Florida. Dose: 0.5 mg/pellet, catalogue no.: E121
The sham operated group: hamsters received the operation but not ovariectomy.
   Method:

47 hamsters weighed 100 g were divided into 6 groups including the positive group, negative group, sham operated and 3 dosage groups (low, medium and high). All hamsters except those in the sham operated group received ovariectomy. A month after the ovariectomy operation, all hamsters were administered intragastrically with drugs and doses indicated above, once a day for consecutive 4 weeks. Blood samples were obtained from the orbital venous sinus, and were subjected to centrifugation at 10,000 rpm for 10 minutes to collect serum. The non-HDL/HDL ratio in serum was analyzed according to See Ho K K W, Leung L K, Chan F L, Huang Y, Chen Z Y (2003).

The hamsters received administration intragastrically for another 4 weeks prior to killing by nitrogen narcosis. The cholesterol level in kidney was measured by gas chromatography.

300 mg of the kidney and 1 mg of the internal standard (IS), stigniastanol (Sigma, S-4297) was homogenized using Polytron homogenizer in 15 ml chloroform-methanol (2:1, v/v) and 3 ml 0.9% saline. The chloroform-methanol phase was transferred to methylation tubes and dried down under a gentle nitrogen steam. After 1 h mild hydrolysis with 5 ml 1N NaOH in 90% ethanol at 90° C., 1 ml of water and 5 ml of cyclohexane were added for cholesterol extraction. The cyclohexane phase was evaporated to dryness under nitrogen, and cholesterol was converted to its TMS-ether derivative by a commercial TMS-reagent (dry pyridine-hexamethyldisilazane-trichlorosilane, 9:3:1, v/v/v, Sil-A reagent, Sigma 1391). After 1h at 60° C., the mixture was dried down under a gentle stream of nitrogen. The TMS-ether derivative was dissolved in 600 μp of hexane, and after centrifugation at 3,000 rpm for 10 min at 4° C., the hexane phase was transferred to a vial for gas-liquid chromatograph (GLC) analysis. The TMS-ether derivative was analyzed in a fused silica capillary column (SAC™-5, 30 m×0.25 mm, i.d.; Supelco, Inc., Bellefonte, Pa., USA) in a Shimadzu GC 14B GLC equipped with a flame-ionization detector (Shimadzu). The column temperature was set at 285° C. and maintained for 20 minutes. The kidney cholesterol (mg/g) was calculated by: [(conc of composition)/(conc of IS)]*[(amount of IS added)/(weight of kidney).

The data of the [non-HDL]/[HDL] ratio in serum and the kidney cholesterol in the hamsters in each group is summarized in Table 12.

TABLE 12

| | | | Kidney |
|---|---|---|---|
| | Number of | serum[non-HDL]/ | cholesterol |
| Group | hamsters | [DL]ratio | (mg/g) |
| Negative control | 8 | 0.58 ± 0.12 | 3.7 ± 0.4 |
| Low dosage | 7 | 0.57 ± 0.25 | 3.4 ± 0.4 |
| (16.6 mg/100 g/day) | | | |
| Medium dosage | 8 | 0.50 ± 0.20 | 3.2 ± 0.1* |
| (33.2 mg/100 g/day) | | | |
| High dosage | 8 | 0.41 ± 0.11* | 3.3 ± 0.3* |
| (66.4 mg/100 g/day) | | | |
| Positive control | 9 | 0.62 ± 0.16 | 3.5 ± 0.4 |
| Sham-operated | 7 | 0.60 ± 0.12 | 3.5 ± 0.3 |

(n = 7˜9, mean ± SD)
*p < 0.05 when compared to negative control

The above results showed that Composition I can increase the HDL level and lower the non-HDL/HDL ratio in serum. It can also lower the cholesterol level in kidney, which means that Composition I is likely to be effective in preventing cardio-vascular diseases.

### EXAMPLE 14

### Effects of Composition I on ERα Transactivation in HepG2 Cells

The experiment is to test the effect of Composition I on the transcriptional activation of estrogen receptor α (ERα). Since estradiol can promote the gene expression of the estrogen response element (ERE), the effect can be measured by observing the luciferase activities of Composition I-treated samples.

The estrogenicity of Composition I can be determined.

See Po L S, Chan Z Y, Tsang D S C & Leung L K (2002).

Reagent:

Sample for test: Composition I was dissolved in saline at concentrations 10⁻⁹ M, 10⁻⁸ M, 10⁻⁷ M, 10⁻⁶ M, 10⁻⁵ M
Sample for control: 17-β-estradiol, purchased from Sigma Chemical Co. (St. Louis, Mo., USA)
ERα expression vector: primers containing restriction sites were used to amplify the full cds of ERα from cDNA of MCF-7. The PCR products were then digested and ligated into pcDNA3.1+ and pC1-Neo respectively. The ligated products were then transformed in DH5α, and mini-prep was then carried out to produce samples of the expression vector. Correct clones were verified by DNA sequencing (Marcogen Ltd., Korea). The ERα expression vector was named pcDNA-er1.
C3-luc: obtained from Dr. D McDonnell, Duke University, Nc, USA
pRL (Renilla luciferase control plasmid): Promega
HepG2 cells: Promega HB8065
Luciferase Assay System: Promega E1980
   Apparatus
24-well culture plates: Iwaki IW3820-024N
   Method:

HepG2 cells were seeded on 24-well culture plates. Twenty-four hours later, the cells were co-transfected with ERα expression vector, C3-luc and pRL (Renilla luciferase control plasmid). After additional 24 hours, the cells were treated with Composition I at concentrations indicated above. The cells were then lysed and assayed to detect the luciferase activities. The effects of Composition I at different concentrations on ERα transactivation in HepG2 cells are summarized in Table 13.
Log10 (Normalized luciferase activity)=luciferase reading (C3-luc)/luciferase reading (pRL)

TABLE 13

| concentration of | Log10 (Normalized luciferase |
|---|---|
| Composition I | activity) |
| 10⁻⁹ M | 0.108 ± 0.0023 |
| 10⁻⁸ M | −0.065 ± 0.0227 |
| 10⁻⁷ M | 0.244 ± 0.0080 |
| 10⁻⁶ M | 0.444 ± 0.0193 |
| 10⁻⁵ M | 0.971 ± 0.0443 |

(n = 3, Mean ± SD)

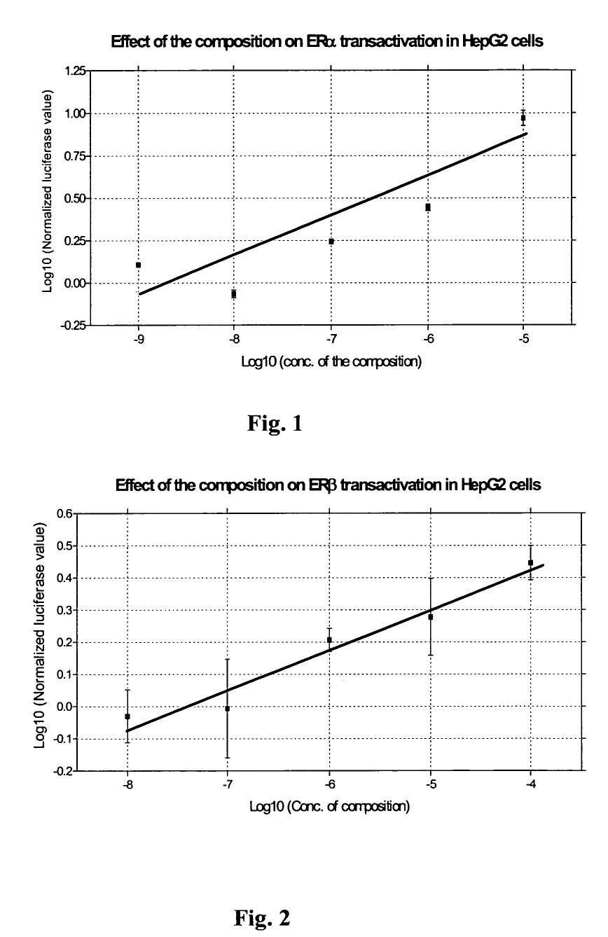

The results showed that Composition I alone induced the transcriptional activation of ERα and the effects were positively correlated with the concentration of Composition I. See FIG. 1.

### EXAMPLE 15

### Effects of Composition I on ERβ Transactivation in HepG2 Cells

The following procedures were carried out as in Experiment 14, except ERβ expression vector were used.

The ERβ expression vector (pC1Neo-ER2) was generated from the ERβ full cds using the procedures described for cloning ERα expression vector.

Method:

HepG2 cells were seeded in 24-well culture plates. Twenty-four hours later, the cells were co-transfected with ERβ expression vector, C3-luc and pRL (Renilla luciferase control plasmid). After additional 24 hours, the cells were treated with various concentrations of Composition I. The cells were then lysed and assayed to detect the luciferase activities. The effects of Composition I on ERβ transactivation in HepG2 cells is summarized in Table 14.

TABLE 14

| concentration of | Log10 (Normalized luciferase |
|---|---|
| Composition I | activity) |
| 10⁻⁸ M | −0.030 ± 0.0823 |
| 10⁻⁷ M | −0.006 ± 0.1530 |
| 10⁻⁶ M | 0.207 ± 0.0357 |
| 10⁻⁵ M | 0.278 ± 0.1197 |
| 10⁻⁴ M | 0.446 ± 0.0536 |

(n = 3, Mean ± SD)

The results showed that Composition I alone induced the transcriptional activation of ERβ and the effects were positively correlated with the concentration of Composition I. See FIG. 2.

### EXAMPLE 16

### Effects of Composition I on Estradiol-Induced ERα Transactivation in HepG2 Cells

With the same reference, reagent, and apparatus in Example 14, the following procedures were carried out:

Method:

HepG2 cells were seeded in 24-well culture plates. Twenty-four hours later, the cells were co-transfected with ERα or ERβ expression vector, C3-luc and pRL (Renilla luciferase control plasmid). Another 24 hours later, the cells were treated with 0 ng/ml, 100 ng/ml or 10 μg/ml of Composition I and 10⁻¹²M to 10⁻⁶M estrdiol. Twenty-four hours later, the cells were then lysed and assayed to detect the luciferase activities. The effects of Composition I on estrdiol induced ERα transactivation in HepG2 cells are summarized in Table 15.

TABLE 15

| | Log10 (luciferase activity) at various | | |
|---|---|---|---|
| Concentration | concentrations of Composition I | | |
| of Estradiol | 0 ng/ml | 100 ng/ml | 10 ug/ml |
| 10⁻¹² M | 0.264 ± 0.0413 | 0.353 ± 0.0528 | 1.208 ± 0.0598 |
| 10⁻¹¹ M | 0.648 ± 0.0490 | 0.901 ± 0.1169 | 1.298 ± 0.1426 |
| 10⁻¹⁰ M | 4.475 ± 0.3530 | 4.593 ± 0.0924 | 4.712 ± 0.4222 |
| 10⁻⁹ M | 6.370 ± 0.3600 | 5.141 ± 0.4072 | 5.605 ± 0.3876 |
| 10⁻⁸ M | 6.212 ± 0.8330 | 5.998 ± 0.1138 | 5.582 ± 0.2159 |
| 10⁻⁷ M | 6.665 ± 0.4690 | 7.039 ± 0.4109 | 7.835 ± 1.0874 |
| 10⁻⁶ M | 8.230 ± 0.8784 | 7.451 ± 1.5420 | 7.574 ± 1.1859 |

(n = 3)Mean ± SD

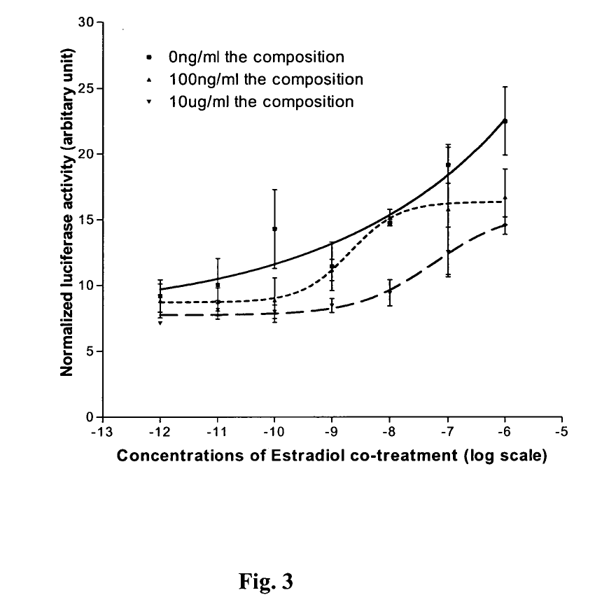

The results showed that Composition I dose not interfere estradiol-induced ER transactivation. See FIG. 3.

### REFERENCES

(1) Benzie I F and Szeto Y T (1999). J. Agric. Food Chem. 47:633-636

(2) Buege J A and Aust S D (1978). Methods in Enzymol.52: 302-310

(3) Chan P T, Fong W P, Cheung Y L, Huang Y, Ho K K W and Chen Z Y. (1999). Journal of Nutrition. 129:1094-1101

(4) Chen Q (1996). Methodology of Pharmacological Research of TCM. People's Medical Publishing House: First Edition

(5) Gu Z P, et al. (1990). Reproductive Pharmacology Anhui Educational publishing House, 1990:57-64

(6) Ho K K W, Leung L K, Chan F L, Huang Y, Chen Z Y (2003). Soy leaf lowers the ration of non-HDL to HDL cholesterol in hamsters. J. Agric. Food Chem.; 51 (16):4554-4558

(7) Index of Traditional Chinese Medicine Innovation Drug Regulatory Department of Ministry of Health People's Republic of China (Pharmacy Pharmacology Toxicology), (1996); 60-61

(8) Instruction of Toxic Research of Chemical Medicine Administration; trial version.

(9) Po L S, Chan Z Y, Tsang D S C & Leung L K (2002). Cancer Letter 187: 33-40

(10) Puhl H., Waege G and Esterbauer H. (1994). Methods in Enzymol. 233: 425-441

(11) W. G. Experimental Direction of Pharmacology-New Medicine Discovery and Pharmacological Evaluation Science Press, 2001:1831-1833

(12) Xu S Y (1991). Methodology of Pharmacological Experiment. People's Medical Publishing House: Second Edition

(13) Xu S Y, et al. (2003). Methodology of Pharmacological Experiment. People's Medical Publishing House: Second Edition, 1537-1540

(14) Yang J, Wang J, et al. (2000). Improvement effects of total paeony glycoside on ability of learning and memory in model mice induced by drugs. Chinese pharmacological Bulletin, 16 (1):46-49

(15) Zhang J T. (1998). Modern Methods of Pharmacological Experiment. Joint Publishing House of Peking Medical University and Pekin Union Medical College

(16) Zhu X D, Tang X C. (1998). Improvement effects of Huperzine A and B on memory impairment of mice. Chinese Pharmacological Bulletin; 9 (6):492-497

What is claimed is:

1) A pharmaceutical composition comprising the extracts of prepared Rehmannia glutinosa Libosch., Epimedium brevicornum Maxim., Cornus officinalis Sieb. et Zucc., Dioscorea opposita Thunb., Lycium barbarum L., and prepared Glycine max (L.) Merr.

2) The pharmaceutical composition according to claim 1, with the proviso that said composition is in the absence of the herbs selected from a group consisting of Poria Cocos (Schw.) Wolf, Paeonia suffruticosa Andr., Cuscuta chinensis Lam., Alisma orientalis (Sam.) Juzep., Codonopsis pilosula (Franch.) Nannf., Bupleurum chinense DC., Glycyrrhiza uralensis Fixch., Pinellia ternate (Thunb.) Breit., Scutellaria baicalensis Georgi, Leonurus japonicus Houtt., prepared Glycyrrhiza uralensis Fixch. or a combination there from.

3) The pharmaceutical composition according to claim 1, wherein said Radix Rehmanniae is Shu Di Huang, said Epimedium brevicornum Maxim is Yin Yang Huo, said Cornus officinalis Sieb. et Zucc. is Shan Zhu Yu, said Dioscorea opposita Thunb. is Shan Yao, said Lycium barbarum L. is Gou Qi Zi, and said Glycine max (L.) Merr. is Dan Dou Chi.

4) The pharmaceutical composition according to claim 3, consisting essentially of the extracts of Shu Di Huang, Yin Yang Huo, Shan Zhu Yu, Shan Yao, Gou Qi Zi, and Dan Dou Chi.

5) The pharmaceutical composition according to claim 3, consisting of the extracts of Shu Di Huang, Yin Yang Huo, Shan Zhu Yu, Shan Yao, Gou Qi Zi, and Dan Dou Chi.

6) The pharmaceutical composition according to claim 3, wherein the extracts are made from the following materials in weight portion:
Shu Di Huang: 4.5-35.5%
Yin Yang Huo: 5.5-24.5%
Shan Zhu Yu: 1.0-25.2%
Shan Yao: 0.5-26.0%
Gou Qi Zi: 3.0-24.0%
Dan Dou Chi: 3.5-22.5%.

7) The pharmaceutical composition according to claim 3, wherein the extracts are made from the following materials in weight portion:
Shu Di Huang: 15-25 portions
Yin Yang Huo: 8-17 portions
Shan Zhu Yu: 5-20 portions
Shan Yao: 10-24 portions
Gou Qi Zi: 10-25 portions
Dan Dou Chi: 9-22 portions.

8) The pharmaceutical composition according to claim 3, wherein the extracts are made from the following materials in weight portion:
Shu Di Huang: 18-22 portions
Yin Yang Huo: 10-13 portions
Shan Zhu Yu: 8-12 portions
Shan Yao: 12-17 portions
Gou Qi Zi: 13-17 portions
Dan Dou Chi: 14-18 portions.

9) The pharmaceutical composition according to claim 3, wherein the extracts are made from the following materials in weight portion:
Shu Di Huang: 19.6 portions
Yin Yang Huo: 11.2 portions
Shan Zhu Yu: 10.4 portions
Shan Yao: 14.0 portions
Gou Qi Zi: 14.4 portions
Dan Dou Chi: 15.4 portions.

10) A method of preparing the pharmaceutical composition of claim 3, comprising providing the extracts of Shu Di Huang, Yin Yang Huo, Shan Zhu Yu, Shan Yao, Gou Qi Zi, and Dan Dou Chi.

11) The method according to claim 10, wherein the portions of the materials are as follows:
Shu Di Huang: 15-25 portions
Yin Yang Huo: 8-17 portions
Shan Zhu Yu: 5-20 portions
Shan Yao: 10-24 portions
Gou Qi Zi: 10-25 portions
Dan Dou Chi: 9-22 portions.

12) The method according to claim 10, wherein the portions of the materials are as follows:
Shu Di Huang: 18-22 portions
Yin Yang Huo: 10-13 portions
Shan Zhu Yu: 8-12 portions
Shan Yao: 12-17 portions
Gou Qi Zi: 13-17 portions
Dan Dou Chi: 14-18 portions.

13) The method according to claim 10, wherein the portions of the materials are as follows:
Shu Di Huang: 19.6 portions
Yin Yang Huo: 11.2 portions
Shan Zhu Yu: 10.4 portions
Shan Yao: 14.0 portions
Gou Qi Zi: 14.4 portions
Dan Dou Chi: 15.4 portions.

14) The method according to claim 10, further comprising the following steps:
(a) providing a mixture comprising herbs of Shu Di Huang, Yin Yang Huo, Shan Zhu Yu, Shan Yao, Gou Qi Zi, and Dan Dou Chi;
(b) extracting the mixture with water, obtaining a first extract solution;
(c) concentrating the first extract solution, forming a concentrated extract solution;
(d) extract the concentrated extract solution with ethanol to obtain a second extract solution and precipitation; and
(e) concentrating said second extract solution.

15) The method according to claim 14, wherein step (b) comprises decocting the mixture in water twice at 90-100° C., 4 hours each time.

16) The method according to claim 14, wherein step (c) comprises heating the first extract solution at 80° C. to a relative density of 1.1-1.3.

17) The method according to claim 14, wherein said ethanol is 70-100% (v/v).

18) The method according to claim 14, wherein the concentrating in step (e) comprises concentrating said second extract solution to a relative density of 1.1-1.5.

19) A method of treatment of menopausal syndromes, comprising administration of a therapeutically effective amount of the pharmaceutical composition of claim 1.

20) Use of the pharmaceutical composition of claim 1 in the preparation of a medicament for the treatment of menopausal syndromes.

21) A nutritional supplement comprising the pharmaceutical composition of claim 1.

22) A formulation for treating depression comprising the pharmaceutical composition of claim 1, wherein the concentration of said composition ranges from 1% to 100% by weight.

23) The formulation according to claim 22, wherein the extracts are made from the following materials in weight portion:
Shu Di Huang: 15-25 portions
Yin Yang Huo: 8-17 portions
Shan Zhu Yu: 5-20 portions
Shan Yao: 10-24 portions
Gou Qi Zi: 10-25 portions
Dan Dou Chi: 9-22 portions.

24) The formulation according to claim 22, wherein the extracts are made from the following materials in weight portion:
Shu Di Huang: 18-22 portions
Yin Yang Huo: 10-13 portions
Shan Zhu Yu: 8-12 portions
Shan Yao: 12-17 portions
Gou Qi Zi: 13-17 portions
Dan Dou Chi: 14-18 portions.

25) The formulation according to claim 22, wherein the extracts are made from the following materials in weight portion:
Shu Di Huang: 19.6 portions
Yin Yang Huo: 11.2 portions
Shan Zhu Yu: 10.4 portions
Shan Yao: 14.0 portions
Gou Qi Zi: 14.4 portions
Dan Dou Chi: 15.4 portions.
